SEQUENCE LISTING (6 sequences)

SEQ ID NO: 1 - protein, 357 aa, Homo sapiens
MAPRRVRSFLRGLPALLLLLLFLGPWPAASHGGKYSREKNQPKPSPKRESGEEFRMEKLNQLWEKAQRLHLPPVRLAELHADLKIQERDELAWKKLKLDGLDEDGEKEARLIRNLNVILAKYGLDGKKDARQVTSNSLSGTQEDGLDDPRLEKLWHKAKTSGKFSGEELDKLWREFLHHKEKVHEYNVLLETLSRTEEIHENVISPSDLSDIKGSVLHSRHTELKEKLRSINQGLDRLRRVSHQGYSTEAEFEEPRVIDLWDLAQSANLTDKELEAFREELKHFEAKIEKHNHYQKQLEIAHEKLRHAESVGDGERVSRSREKHALLEGRTKELGYTVKKHLQDLSGRISRARHNEL

SEQ ID NO: 2 - protein, 15 aa, Homo sapiens
RVIDLWDLAQSANLT

SEQ ID NO: 3 - RNA, 58 nt, synthetic construct
  misc_feature 1..58: note = Description of Artificial Sequence: Syntheticoligonucleotide
  source 1..58: mol_type = other RNA
ccgggctgaacacattctttggtaactcgagttaccaaagaatgtgttcagctttttg

SEQ ID NO: 4 - protein, 491 aa, Homo sapiens
PCKVNNGGCSNLCLLSPGGGHKCACPTNFYLGSDGRTCVSNCTASQFVCKNDKCIPFWWKCDTEDDCGDHSDEPPDCPEFKCRPGQFQCSTGICTNPAFICDGDNDCQDNSDEANCDIHVCLPSQFKCTNTNRCIPGIFRCNGQDNCGDGEDERDCPEVTCAPNQFQCSITKRCIPRVWVCDRDNDCVDGSDEPANCTQMTCGVDEFRCKDSGRCIPARWKCDGEDDCGDGSDEPKEECDERTCEPYQFRCKNNRCVPGRWQCDYDNDCGDNSDEESCTPRPCSESEFSCANGRCIAGRWKCDGDHDCADGSDEKDCTPRCDMDQFQCKSGHCIPLRWRCDADADCMDGSDEEACGTGVRTCPLDEFQCNNTLCKPLAWKCDGEDDCGDNSDENPEECARFVCPPNRPFRCKNDRVCLWIGRQCDGTDNCGDGTDEEDCEPPTAHTTHCKDKKEFLCRNQRCLSSSLRCNMFDDCGDGSDEEDCSIDPKLT

SEQ ID NO: 5 - DNA, 20 bp, synthetic construct
  misc_feature 1..20: note = Description of Artificial Sequence: Syntheticoligonucleotide
  source 1..20: mol_type = other DNA
gactgaggaggcggaaacaa

SEQ ID NO: 6 - protein, 14 aa, Homo sapiens
RGEVQAMLGQSTEE

(19) United States
(12) Patent Application Publication
(10) Pub. No.: US 2024/0247055 A1
(43) Pub. Date: Jul. 25, 2024

(54) Inhibition of Tau Propagation

(71) Applicant: The Regents of the University of California, Oakland, CA (US)

(72) Inventors: Kenneth Kosik, Santa Barbara, CA (US); Jennifer Rauch, Santa Barbara, CA (US)

(73) Assignee: The Regents of the University of California, Oakland, CA (US)

(21) Appl. No.: 18/439,648

(22) Filed: Feb. 12, 2024

Related U.S. Application Data
(63) Continuation of application No. 17/511,343, filed on Oct. 26, 2021.
(60) Provisional application No. 63/105,667, filed on Oct. 26, 2020.

(51) Int. Cl. C07K 16/18

(52) U.S. Cl. CPC C07K 16/18; C07K 2317/20; C07K 2317/76

(57) ABSTRACT

Provided are novel methods of inhibiting the propagation of pathological forms of tau between cells of the central nervous system by administration of an inhibitor of LRP1. LRP1 was discovered to facilitate the endocytosis of pathological forms of tau which enables the propagation of tau aggregates and resulting neurodegeneration. These methods enable the prevention and treatment of various tauopathies, such as Alzheimer's, chronic traumatic encephalopathy, corticobasal degeneration, and others.

### CROSS-REFERENCE TO RELATED APPLICATIONS

This application is a continuation of U.S. application Ser. No. 17/511,343 entitled “Inhibition of Tau Propagation,” filed Oct. 26, 2021, which claims the benefit of priority to U.S. Provisional Application Ser. No. 63/105,667 entitled “Inhibition of Tau Propagation,” filed Oct. 26, 2020, the contents which are hereby incorporated by reference in their entirety.

### STATEMENT REGARDING FEDERALLY SPONSORED RESEARCH OR DEVELOPMENT

This invention was made with government support under grant numbers AG064116 and NS100717 awarded by The National Institutes of Health. The government has certain rights in the invention.

### STATEMENT REGARDING SEQUENCE LISTING

The instant application contains a Sequence Listing which has been submitted electronically in ASCII format and is hereby incorporated by reference in its entirety. Said ASCII copy, created on Oct. 25, 2021, is named UCSB034PCT_SL.txt and is 8,439 bytes in size.

### BACKGROUND OF THE INVENTION

The spread of protein aggregates during disease progression is a common theme underlying many neurodegenerative diseases. The microtubule-associated protein tau has a central role in the pathogenesis of several forms of dementia known as tauopathies—including Alzheimer's disease, frontotemporal dementia and chronic traumatic encephalopathy. Progression of these diseases is characterized by the sequential spread and deposition of protein aggregates in a predictable pattern that correlates with clinical severity. This observation and complementary experimental studies have suggested that pathologic forms tau can spread in a prion-like manner, by passing to naive cells in which it templates misfolding and aggregation. However, although the propagation of tau has been extensively studied, the underlying cellular mechanisms by which aberrant tau spreads remains poorly understood.

Accordingly, there is a need in the art for a deeper understanding of tau propagation mechanisms. There is a need in the art for interventions to slow or halt pathologic spread of tau in neurons. Finally, there is a need in the art for novel methods of treating tauopathies.

### SUMMARY OF THE INVENTION

The inventors of the present disclosure have determined that the uptake of pathologic tau, such as misfolded monomeric tau and tau fibrils, is mediated by the enzyme low density lipoprotein receptor related protein 1 (LRP1). Herein it is demonstrated that LRP1 achieves the endocytosis of pathological forms of tau into naïve cells, underlying the subsequent spread of toxic tau aggregates in cells of the central nervous system. Furthermore, the inventors of the present disclosure have demonstrated that inhibition of LRP1-mediated tau uptake by various agents can halt the intercellular spread of pathological tau conformations, providing the art with a novel method of treating a number of neurodegenerative diseases.

In a first aspect, the scope of the invention encompasses a method of inhibiting the uptake of pathological forms of tau by cells of the central nervous system by inhibition of LRP1.

In another aspect, the scope of the invention encompasses a method of inhibiting the spread of pathological forms of tau between cells by inhibition of LRP1

In another aspect, the scope of the invention encompasses a method of treating tauopathies by inhibition of LRP1.

The various element of the invention are describe in detail next.

### BRIEF DESCRIPTION OF THE DRAWINGS

FIG. 1. Uptake of a disease-relevant mutant of tau and of phosphorylated tau are inhibited by LRP1 knockdown. Uptake of phosphorylated (p2N4R) or mutated (2N4RP301L) full length tau in H4i cells (n=8). WT=Wild Type. NT sgNRA=cells treated with control non-targeting CRISPR construct. LRP1 sg RNA=cells treated with LRP1 targeting CRISPR construct. One-way ANOVA with Tukey's method, two-sided was performed to determine significance, ****=p-value<0.0001.

FIGS. 2A and 2B. FIG. 2A: Uptake of 2N4R tau with RAP or lysine-capped RAP control competition. FIG. 2B: Uptake of 2N4R tau with RAP or mtRAP competition (mtRAP is mutant RAP with point mutations of key residues, K256A, K270A, necessary for its interaction with LRP1.

FIG. 3. FIG. 3 demonstrates Tau and transferrin uptake by cells mock treated or overexpressing ApoE (n=3) isoforms. Incubation of H4 cells in ApoE conditioned medium significantly reduced uptake of full-length tau, yet failed to reduce transferrin uptake. One-way ANOVA with Tukey's method, two-sided was performed to determine significance, ****=p-value<0.0001.

FIG. 4. FIG. 4 depicts as schematic diagram of LRP1 ectodomains.

FIGS. 5A and 5B. FIG. 5A: Uptake of 2N4R monomers and oligomers in CRISPRi iPS-derived neurons (iPSNs), n=10. FIG. 5B: Uptake of 2N4R tau (WT vs. mtRAP; p-value=>0.9999) or Tfn (ANOVA; p-value=0.7815) in the presence of RAP or mtRAP (n=7). All experiments were performed over three independent experiments and normalized to WT uptake (100%). Data expressed as mean±s.d. One-way ANOVA with Tukey's method, two-sided was performed to determine significance. Displayed is the multiple comparison against WT, ns=not-significant, ****=p-value<0.0001. Comparison to NT sgRNA resulted in the same level of statistical significance.

FIGS. 6A, 6B, and 6C. FIGS. 6A, 6B, and 6C depict tau spread in vivo, in mice treated with PBS buffer control, scramble shRNA control or LRP1 shRNA. FIG. 6A: Quantification of total number of hTau⁺/GFP⁻ cells/mm² of PBS, scramble shRNA or LRP1 shRNA mice (PBS vs. scramble shRNA; p-value=0.1582). FIG. 6B: Quantification of total number of GFP⁺ cells/mm² (ANOVA; p-value=0.8007). FIG. 6C: Quantification of ipsilateral hippocampal hTau⁺/GFP⁻ cells/mm² in PBS, scramble shRNA or LRP1 shRNA mice (ANOVA; p-value=0.0692).

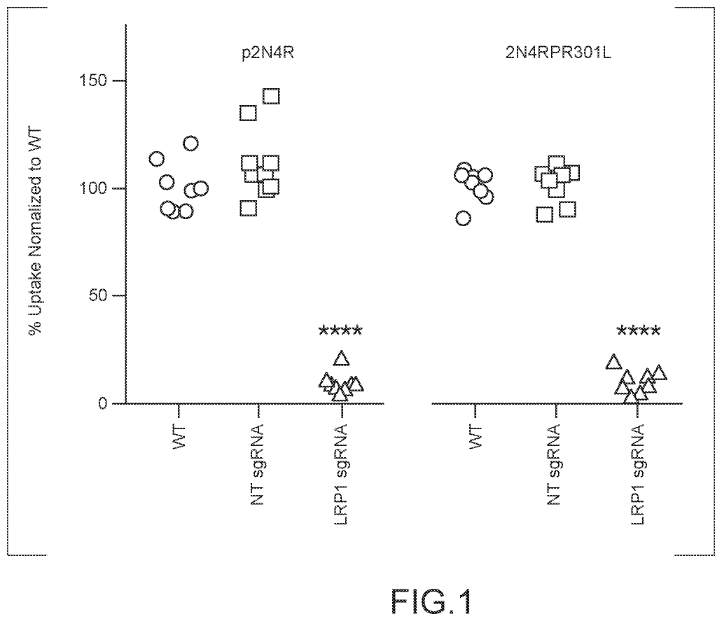

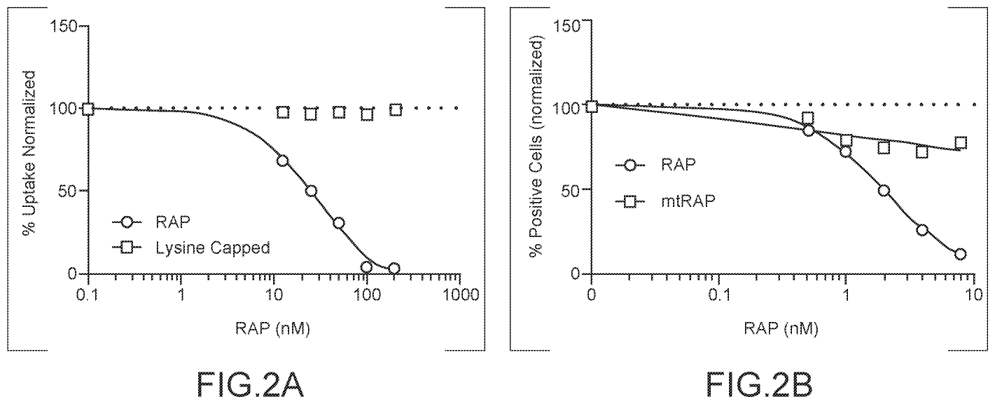

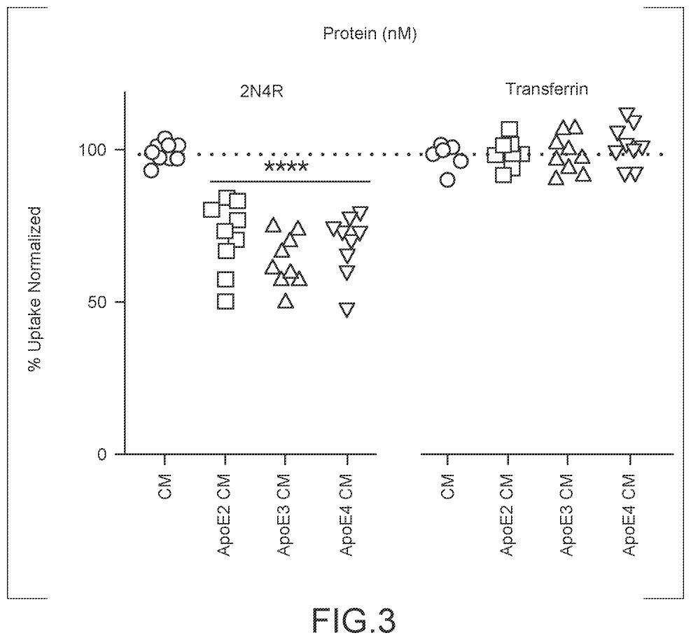

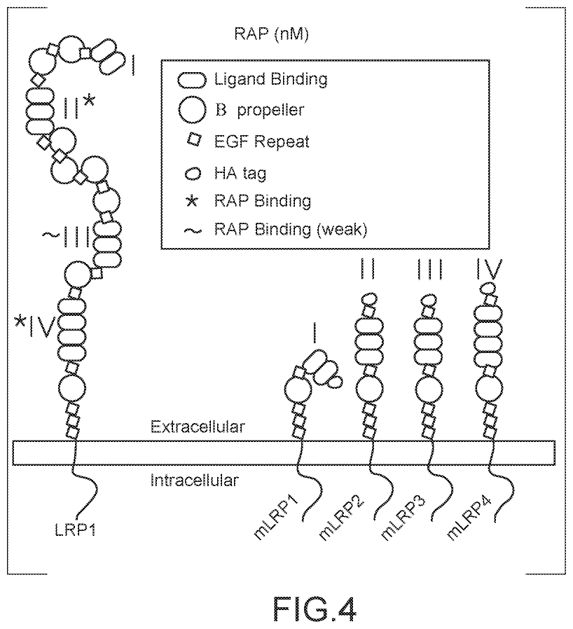

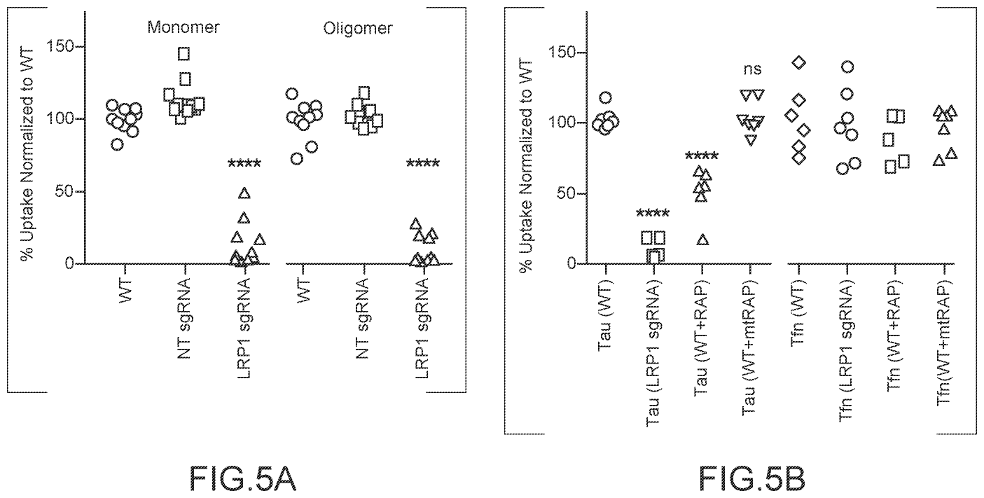

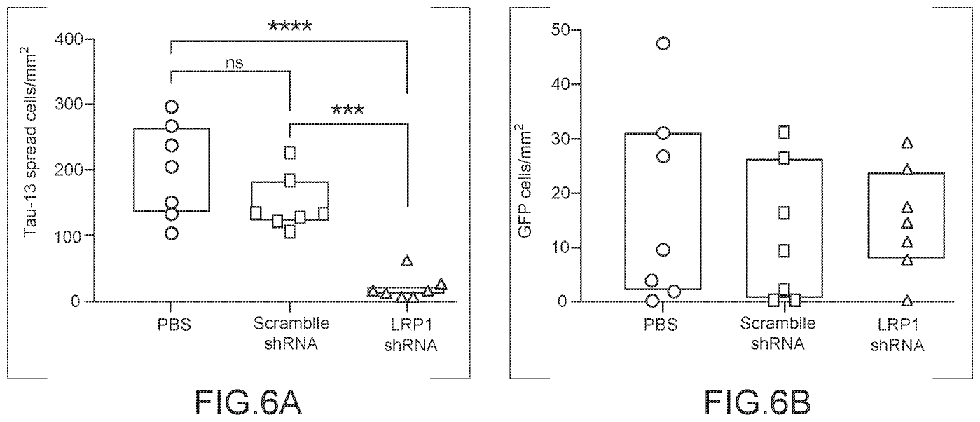

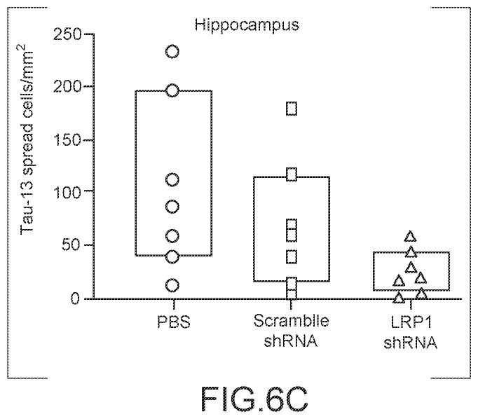

### DETAILED DESCRIPTION OF THE INVENTION

The scope of the invention encompasses methods that provide the art with novel means of inhibiting the uptake of pathological forms of tau, inhibiting the spread of pathologic tau between cells, and treating tauopathies. These therapeutic outcomes are achieved by inhibition of LRP1.

In a first aspect, the scope of the invention encompasses a method of inhibiting the uptake of pathological tau by cells of the central nervous system cells of a subject, comprising the administration to the subject of a therapeutically effective amount of an inhibitor of LRP1. In a related aspect, the scope of the invention encompasses an inhibitor of LRP1 for use in a method of inhibiting the uptake of pathological tau by cells of the central nervous system cells of a subject. In another aspect, the scope of the invention encompasses a method of utilizing an inhibitor of LRP1 to make a medicament for the inhibition of uptake of pathological tau by cells of the central nervous system.

In one aspect, the scope of the invention encompasses a method of inhibiting the propagation of pathological tau in the central nervous system of a subject, comprising the administration to the subject of a therapeutically effective amount of an inhibitor of LRP1. In a related implementation, the scope of the invention encompasses an inhibitor of LRP1 for use in a method of inhibiting the propagation of pathological tau in the central nervous system of a subject. In another aspect, the scope of the invention encompasses a method of utilizing an inhibitor of LRP1 to make a medicament for the inhibition of pathological tau propagation in the central nervous system.

In one aspect, the scope of the invention encompasses a method of treating a tauopathy in a subject, comprising the administration to the subject of a therapeutically effective amount of an inhibitor of LRP1. In a related aspect, the scope of the invention encompasses an inhibitor of LRP1 for use in a method of treating a tauopathy in a subject. In yet another implementation, the scope of the invention encompasses the utilization of an inhibitor of LRP1 to make a medicament for the treatment of a selected tauopathy.

Subjects. As used herein, a “subject” may comprise any animal. In a primary embodiment, the subject is a human, for example, a human patient. In other embodiments, the subject may comprise a test animal, veterinary subject, or other non-human animal, for example, a mouse, rat, dog, cat, cow, horse, pig, or non-human primate. In some implementations, the subject comprises cultured cells, as in an experimental or drug screening process.

In some implementations, the subject is a subject having a tauopathy, for example diagnosed with a tauopathy or suspected of having a tauopathy by presentation of one more clinical or cognitive indicators of tauopathy. In some implementations, subject is a subject at risk of a tauopathy, for example, at risk by having one or more genetic mutations associated with a tauopathy, having a family history of tauopathy, or being an aged subject. Exemplary aged subjects include human subjects of at least 45 years of age, at least 50 years of age, at least 60 years or age, or at least 65 years of age.

Therapeutically Effective Amount. As used herein, a “therapeutically effective amount” means an amount sufficient to promote a measurable biological response, measurable therapeutic effect, or other detectable measure of efficacy. In one embodiment, a therapeutically effective amount is an amount sufficient to measurably inhibit the uptake of pathologic tau by one or more selected cell types of the central nervous system. In one embodiment, the therapeutically effective amount is an amount sufficient to measurably inhibit the propagation of pathologic tau between cells of the central nervous system. In one embodiment, the therapeutically effective amount is a an amount sufficient to measurably treat a neurodegenerative disorder mediated by the accumulation of pathologic tau.

The LRP1 inhibitor may be administered at any effective dosage. One of skill in the art may determine the dosage by taking into account the physical, chemical, and pharmacological (e.g. ADMET) properties of the administered pharmaceutical composition, the route of administration, and the therapeutic need. Exemplary dosages may include cumulative daily dosages of 10 ng to 50 mg/kg body weight per day, for example: 10 ng, 50 ng, 100 ng, 200 ng, 500 ng, 1 μg 5 μg, 10 μg, 15 μg, 20 μg, 25 μg, 30 μg, 40 μg, 50 μg, 60 μg, 70 μg, 80 μg, 90 μg, 100 μg, 200 μg, 300 μg, 400 μg, or 500 μg, 1 mg, 5 mg, 10, mg, or more mg per kg body weight per day. Administration may be daily, including multiple daily administrations (e.g. 2, 3, 4, 5, or more), or multiple times per week.

Pathological Tau. In various embodiments, the scope of the invention encompasses methods of halting the uptake and propagation of pathological tau in the central nervous system. As used herein, “pathologic tau” means a form of tau, i.e. a tau conformer, which acts as a template to recruit native tau into toxic or otherwise pathological aggregates, or which otherwise is detrimental to one or more functions of a cell of the central nervous system. Pathologic tau may exert its detrimental effects on cells by direct toxicity or by disrupting normal tau function, such as disrupting microtubule stabilization. As known in the art, different forms of tauopathy are associated with different types of pathologic tau conformations.

In various embodiments, pathologic tau may comprise hyperphosphorylated tau protein, tau aggregates, tau oligomers, tau fibrils, and higher-order aggregates of tau. Exemplary pathological forms if tau include: the full-length soluble monomeric tau; the tau 2N4R isoform; the 0N3R isoform; the 0N4R isoform; the 1N3R isoform; the 1N4R isoform; the 2N3R isoform; or tau fragments that contain microtubule binding repeat region such as K18 and K19 repeats. In one embodiment, the pathologic tau comprises a tau seed. A tau seed comprises a pathological form of tau and may comprise a disease-specific form of tau oligomer, for example a misfolded monomer or small fibril capable of seeding the formation of aggregates. For example, the pathologic Tau may comprise a disease specific seed form as known in the art, for example, as are known for Alzheimer's disease, traumatic encephalopathy, corticobasal degeneration, Pick's Disease, progressive supranuclear palsy, globular glial tauopathy, and argyrophilic grain disease.

Tau uptake. The scope of the invention encompasses methods of inhibiting the uptake of pathological tau by cells of the central nervous system. “Uptake” as used herein encompasses the uptake of extracellular pathological tau into a cell of the central nervous system, wherein the uptake is endocytosis mediated by LRP1. Cells of the central nervous system include, for example, any of neurons, glial cells, astrocytes, oligodendrocytes, and other cells of the brain or CNS.

Tau Propagation. The scope of the invention encompasses methods of inhibiting the propagation of pathological tau between cells of the central nervous system. “Propagation” as used herein encompasses the spread of extracellular pathological tau between cells of the central nervous system, wherein the uptake of the pathological tau, for example, to naïve cells, is mediated by LRP1. “Inhibition of propagation” as used herein, encompasses any inhibition, e.g. reducing, slowing, halting, ablating or otherwise disrupting any cell-to-cell transfer of pathological tau or the propagation of pathological tau aggregations from one region of the CNS to another. Tau propagation often proceeds in a predictable manner specific to a selected tauopathy. For example, in Alzheimer's disease, it is thought that neurofibril tangles of tau first develop in the transentorhinal cortex and subsequently migrate to the limbic system, ultimately affecting the isocortical regions. In this context, inhibition of tau propagation may comprise any slowing, reduction, or ablation of the spread of pathological tau deposits in a stereotypical progression associated with a particular disease type.

Tauopathies. In one aspect, the scope of the invention encompasses the treatment of a tauopathy by inhibition of LRP1. As used herein, “treatment” encompasses any therapeutic effect with regards to a selected condition. In various aspects, treatment may encompass prevention of a selected condition, slowing the progression of the selected condition, ameliorating the symptoms of a selected condition, reversing the progression of a selected condition, curing the symptoms of a selected tauopathic condition, improving cognitive function, slowing the accumulation of pathological tau aggregates in the central nervous system, reducing cell death, improving neuromotor function, or reducing the amount of pathologic tau in selected cells, components, or organs of the central nervous system.

In one embodiment, the tauopathy is Alzheimer's disease. Alzheimer's disease may be characterized by aggregates of both 3R and 4R tau isoforms, i.e. tau isoforms having three or four repeats, respectively, in the tau microtubule-binding domain. In Alzheimer's, tau aggregates manifest as tau neurofibrillary tangles (NFTs), neuropil threads, and plaque-associated neurites. In Alzheimer's, the spread of pathologic tau inclusions follows a predictable progression across areas of the brain.

In one embodiment, the tauopathy is frontotemporal dementia. As used herein, frontotemporal dementia (FTD) encompasses any form of FTD associated with tau dysfunction. Exemplary forms include dementia lacking distinctive histopathology (DLDH), frontotemporal lobar degeneration (FTLD), familial FTD known as hereditary dysphasic disinhibition dementia 2 (HDDD2), and frontotemporal dementia with parkinsonism linked to chromosome 17 (FTDP-17).

In one embodiment, the tauopathy is corticobasal degeneration. Corticobasal degeneration may be characterized by filamentous 4R tau inclusions in neurons and glia, with variable distributions among subjects in frontal, temporal, and parietal lobes and basal ganglia. Pathology may include “ballooned” neurons in the neocortex, intraneuronal basophilic tau inclusions, astrocytic plaques, coiled bodies oligodendrocytes, NFTs in the basal ganglia and brainstem, and neuron loss in the substantia nigra.

In one embodiment, the tauopathy is progressive supranuclear palsy (PSP). PSP may be characterized by filamentous 4R tau inclusions, for example, filamentous tau inclusions in neurons, astrocytes, and oligodendrocytes.

In one embodiment, the tauopathy is Pick disease. Pick disease may be characterized by dense, spherical tau inclusions called Pick bodies. Pick disease pathology may include, for example, Pick bodies in dentate gyrus, ballooned neurons in cortical and subcortical regions, neuron loss, and glial Pick body inclusions in the gray and white matter.

In one embodiment, the tauopathy is argyrophilic grain disease. Argyrophilic grain disease may be characterized by 4R tau inclusions, for example, argyrophilic grains, which are small spindle or comma shaped structures found within dendrites of the cerebral cortex. Argyrophilic grain disease may also encompass tau inclusions such as oligodendrocytic coiled bodies and neuronal pretangles.

In one embodiment, the tauopathy is chronic traumatic encephalopathy (CTE). CTE is associated with repetitive head trauma and may be characterized by presence of hyperphosphorylated tau protein in neurons, astrocytes and cell processes around blood vessels as well as distinctive tau filaments in cortical layers II and III. CTE often follows a progression from isolated focal tau lesions in the cerebral cortex to a widespread tauopathy that involves diffuse cortical and medial temporal lobe regions, likely as a result of tau propagation.

In one embodiment, the tauopathy is primary age-related tauopathy. Primary age-related tauopathy may be characterized by NFTs in the hippocampus and entorhinal cortex.

Other tauopathies include, for example, aging-related tau astrogliopathy (ARTAG); geographically isolated PSP-like tauopathies such as Guam Parkinsonism-dementia complex (PDC) and Guadeloupean parkinsonism; globular glial tauopathy (GGT); and non-specific tauopathies.

### LRP1 Inhibitors.

The inventions disclosed herein encompass the administration of LRP1 inhibitors to a subject. LRP1 is the Low density lipoprotein receptor related protein 1, also known as alpha-2-macroglobulin receptor (A2MR), Apolipoprotein E receptor (APOE-R), or CD91. LRP1 is a large endocytic receptor that binds and mediates the endocytosis of numerous and structurally diverse ligands.

As used herein, “LRP1 inhibitor” means a composition of matter which inhibits the transmembrane transport of tau by LRP1. Inhibition may encompass any reduction in LRP1 tau transport or endocytosis activity, including competitive inhibition, non-competitive inhibition, a reduction in LRP1 expression, a reduction in LRP1 protein abundance, or a reduction in LRP1 tau transport competence or binding ability.

As demonstrated herein, the inventors of the present disclosure have determined that tau endocytosis by LRP1 is primarily facilitated by LRP1 domain 4 and partially by LRP1 domain 2. Accordingly, in one implementation, the LRP1 inhibitor comprises an agent which inhibits LRP1 domain 4 activity, LRP1 domain 2 activity, or both LRP1 domain 4 and domain 2 activity. LRP1 has many ligands and is implicated in a variety of cellular processes. Accordingly, in some implementations, to avoid pleiotropic side effects, it is advantageous to select an LRP1 inhibitor that is selective for inhibiting tau endocytosis and which does not substantially interfere with other processes mediated by LRP1. In one embodiment, the LRP1 inhibitor selectively or preferentially inhibits LRP1 domain 4 activity, LRP1 domain 2 activity, or both LRP1 domain 4 activity and LRP1 domain 2 activity.

RAP and Variants. In a first implementation, the LRP1 inhibitor comprises Alpha-2-macroglobulin receptor-associated protein receptor-associated protein (RAP) or a variant thereof. RAP is a molecular chaperone that binds LRP1 and other low density lipoprotein receptor family members and is integral to their recycling. Exogenous RAP acts as a potent inhibitor to LRP1 binding to LRP1 on the cell surface and preventing ligands from binding to LRP1.

In one implementation, RAP may encompass the human Alpha-2-macroglobulin receptor-associated protein, for example, the protein of SEQ ID NO: 1. In a various implementations, the LRP1 inhibitor is a variant of RAP. As used herein, a “variant” of RAP is a composition having structural similarity to a native RAP sequence, or a subsequence thereof, and which also retains LRP1 inhibiting activity. Variants may include truncations, variants comprising amino acid substitutions, insertions, or deletions. In various embodiments the RAP variant comprises a polypeptide having at least 75%, at least 80%, at least 85%, at least 90%, at least 95% or at least 99% similarity to SEQ ID NO: 1.

In one embodiment, the RAP variant comprises an active fragment of RAP, i.e. a subsequence of the RAP protein that retains the ability to inhibit pathological tau endocytosis by LRP1. In one embodiment, the fragment comprises the RAP D1 and D2 domains, for example, amino acids 1-215 of SEQ ID NO: 1. In one embodiment, the fragment comprises the RAP D3 domain, for example, amino acids 216-323 of SEQ ID NO: 1. In one embodiment, the fragment comprises at least residues 201-210 of SEQ ID NO: 1. In one embodiment, the fragment comprises at least residues 256-270 of RAP, for example, as in the fragment of SEQ ID NO: 2 which comprises 256-270 of RAP.

In one embodiment, the variant comprises a stabilized form of RAP. As part of its recycling functions, RAP dissociates in the acidic environment of the golgi complex, by the destabilization of its domain 3. As known in the art, variants of RAP have been engineered with improved heat and acidic stability. For example, in one embodiment, the RAP variant comprises RAP having one or more mutations selected from the group consisting of Y260C, T297C, H257F, H259F, H268F, and H290F, as described in Prasad et al., 2015, Generation of a Potent Low Density Lipoprotein Receptor-related Protein 1 (LRP1) Antagonist by Engineering a Stable Form of the Receptor-associated Protein (RAP) D3 Domain, J Biol Chem 290: 17262-17268.

In one embodiment, the variant comprises a cyclic form of RAP comprising a covalent bond between two non-consecutive amino acids, which may stabilize the three-dimensional structure of the RAP peptide and/or increase its LRP1 inhibiting activity. In various embodiments, the cyclic variant is a composition disclosed in PCT International Patent Application Number WO2008116171, Cyclic Receptor-Associated Proteins” by Starr and Zankel.

In another implementation, the LRP1 inhibitor is an antibody or antigen-binding fragment thereof which selectively binds to LRP1 and inhibits the tau-endocytosis activity of LRP1. Exemplary anti-LRP1 antibodies include, for example, Abcam, ab-92544, Molecular Innovations catalog number MA-8G1, Sigma Millipore Antibody L2420, Antibody Registry entries AB_11176350, AB_11178593, AB_11172700, Santa Cruz Biotech and Invitrogen Catalog #MA1-27198.

LRP1 Expression Inhibitors. In one embodiment, the LRP1 inhibitor is an agent, such as a nucleic acid construct, which inhibits the expression of the LRP1 gene in a cell of the central nervous system to which it is administered.

In a primary implementation, the LRP1 expression inhibitor is an element of a CRISPR-Cas9 or like system for the targeted knockdown the LRP1 gene. In one embodiment, the LRP1 expression inhibitor comprises the 5′ targeting sequence of a CRISPR guide RNA, the targeting RNA comprising, for example, a 15-25 nucleotide subsequence of the LRP1 gene (either coding or non-coding strand), for example, a 17-20 nucleotide sequence, wherein the sequence is adjacent to a suitable protospacer adjacent motif (PAM site), for example, NGG, or CCN, wherein N is any nucleotide. In one embodiment, the guide RNA comprises a sequence having at least 90%, at least 95%, or at least 99% sequence identity to SEQ ID NO: 5. In one embodiment, the guide sequence is present in an expression vector, such as a plasmid, which codes for the guide RNA sequence, and typically will be co-expressed with an engineered Cas9 protein, for example, a Streptococcus pyognes Cas9 system (combined cRNA:tracrRNA, for example), for example, codon optimized for expression in the target organism, for example, optimized for expression in human cells. SpCas9 variants may also be used with altered PAM site specificities, for example, the D1135E, VRQ, EQR, VRER, xCas9, SpG and SpRY variants, as known in the art. The Cas9 and guide RNA sequences may be placed under the control of a suitable promoter. In one embodiment, the promoter is a promoter for selective or preferential expression in CNS cells, such as neurons. When expressed in the target cells, the guide RNA and Cas9 form a complex that will specifically targeted by the guiding sequence to the miRNA gene, activating Cas9 exonuclease cleavage of the targeted DNA, resulting in a double stranded break about three nucleotides upstream of the adjacent PAM site. Subsequent non-homologous end joining (NHEJ) results in an indel mutation which disrupts the expression of the targeted gene.

In alternative implementations, the LRP1 expression inhibitor may comprises other compositions for the targeted mutagenesis of the LRP1 gene, for example, a zinc finger nuclease (ZNF), or transcription activator-like effector nuclease (TALEN) targeted to the LRP1 gene. In another embodiment, the LRP1 expression inhibitor may comprise a nucleic acid sequence which selectively interferes with transcription or processing of the targeted miRNA such as an antisense construct, short interfering RNA (siRNA), or short hairpin (shRNA) sequence.

In one embodiment the nucleic acid construct is an short hairpin RNA (shRNA) targeting LRP1, for example, under the human synapsin promoter, used to knock down LRP1 in neurons, as described in the Examples. In one embodiment, the LRP1 inhibitor comprises an shRNA sequence such as a nucleic acid comprising at least 90%, at least 95%, or at least 99% sequence identity to SEQ ID NO: 3.

Competitive Inhibitors and LRP1 Ligands. In one implementation, the LRP1 inhibitor is a competitive inhibitor. The competitive inhibitor may be a species that binds one or more conformers of tau, inhibiting endocytosis by competing with LRP1 for tau binding. In another embodiment, the competitive inhibitor is a species that binds LRP1, inhibiting endocytosis by competing with tau for LRP1 binding. In one embodiment, the competitive inhibitor is a polypeptide or peptide mimetic with binding affinity for one or more forms of pathologic tau. In one embodiment, the therapeutic peptide comprises an LRP1 domain or subsequence that binds pathological tau, for example, in one embodiment, the LRP1 inhibitor comprises a soluble fragment of LRP1 domain IV, for example, from amino-acids 3293-3783 of the full length LRP1 protein SEQ ID NO: 1. In one embodiment, the therapeutic peptide is a polypeptide having at least 90%, at least 95%, or at least 99% sequence identity to SEQ ID NO: 4. In one embodiment, the therapeutic peptide is Recombinant Human LRP-1 Cluster IV Fc Chimera Protein, CF, R&D Biosystems Catalog Number 5395-L4-050.

In one embodiment the competitive inhibitor comprises an LRP1 ligand or other species that binds LRP1 and which compete with tau for LRP1 binding. Exemplary LRP1 ligands include apolipoprotein E (apoE), for example, used as an inhibitor as described in Woollet et al., Apolipoprotein E competitively inhibits receptor-dependent low density lipoprotein uptake by the liver but has no effect on cholesterol absorption or synthesis in the mouse, PNAS 92:12500-4. ApoE encompasses various isoforms of ApoE, such as ApoE2, APoE3, and ApoE4, and LRP1 binding variants or fragments thereof. In one embodiment, the competitive inhibitor is ApoE4. In one embodiment, the competitive inhibitor comprises at least amino acids 136 to 150 of ApoE which is the LRP1 binding site. In one embodiment, the competitive inhibitor is a polypeptide comprising SEQ ID NO: 6, which comprises amino acids 136 to 150 of ApoE. In other embodiments, the competitive inhibitor comprises an LRP1 ligand or LRP1-binding domain thereof, selected from the group consisting of: Albumin, Angiotensin 1-7, Angiotensin II, ApoB, ApoE, ApoH, Apoj (Clusterin), ApoM, Aprotinin, Bone morphogenetic protein 4, Ca²⁺, Cathepsin b, Coagulation Factor VIII, Connective tissue growth factor, Cystatin C, Cytochrome C, Epidermal growth factor, Folate binding protein, Frizzled-1, Hemoglobin, Insulin, Lactoferrin, Leptin, Lipoprotein lipase, Metallothionein, Plasminogen, Plasminogen activator inhibitory type 1, Plasminogen activator inhibitory type 1 tissue plasminoegen activator, Plasminogen activator inhibitory type 1 urokinase, Retinol binding protein, Sonic hedgehog protein.

In one embodiment, the therapeutic peptide of the invention encompasses a polypeptide that inhibits LRP1 by being partially endocytosed. In one embodiment, the therapeutic peptide is cathepsin D or variant thereof, for example, as described in DeCroq et al., 2012. Cathepsin D is partly endocytosed by the LRP1 receptor and inhibits LRP1-regulated intramembrane proteolysis. Oncogene 31: 3202-3212.

Small molecules. In one embodiment, the inhibitor of LRP1 is a small molecule inhibitor.

Therapeutic Compositions and Delivery. The LRP1 inhibitor utilized in the methods of the invention may be formulated into therapeutic compositions or medicaments for delivery to CNS target cells by a selected method of administration. Exemplary administration methods include for example, by intracerebroventricular, intraparenchymal, intranasal, or intrathecal injections. In some implementations, convection-enhanced delivery (CED) is utilized to target specific brain structures. Other administration routes that avoid the blood brain barrier may be used, for example, administration nasal administration at the cribriform plate or injection into the subarachnoid space of the spinal cord.

Other potential routes include, for example, intravenous, intra-arterial, intraperitoneal, intrapulmonary, oral, inhalation, intravesicular, intramuscular, intra-tracheal, subcutaneous, intraocular, intrathecal, transmucosal, and transdermal delivery.

The therapeutic compositions of the invention may encompass LRP1 inhibitors combined with, modified with, loaded with, or otherwise combined with compositions that facilitate delivery to target cells of the CNS, including pharmaceutically acceptable excipients, carriers, diluents, release formulations and other drug delivery or drug targeting vehicles, as known in the art. In one embodiment, the formulations comprise, compositions that aid in traversing the blood-brain barrier (BBB). Exemplary formulation methods include cationization of therapeutic agents, fusion or functionalization of therapeutic agents with cell-penetrating peptides (such as trans-activating transcriptional activator, penetratin, and the Syn-B), fatty acid acylation of therapeutic agents, PEGylation. In some embodiments, the therapeutic agent is conjugated to an antibody or antibody fragment which aids in crossing the BBB or targeting the brain, such as antibodies against the insulin receptor, gliofibrillar acid protein or brain specific α2-glycoprotein.

In one implementation, the LRP1 inhibitor is formulated for particle delivery, wherein the therapeutic agent is encapsulated, conjugated, loaded, or otherwise delivered by particles, such as liposomes, amphiphilc block copolymers (for example, pluronic block copolymers), polyion complex micelle, PLGA nanoparticles, poly(butylcyanoacrylate) nanoparticles, polyion complexes, PEG-silica, bolaamphiphilies, chitosan, PEG-polylactide, PEG-poly(ε-caprolactone) PLA-D-α-Tocopheryl polyethylene glycol succinate. In other implementations, the therapeutic agents are loaded onto, or expressed by cellular delivery vehicles, such as functionalized macrophages or macrophage expression vectors. In one embodiment, the LRP1 inhibitor is formulated with a carrier that can be selectively activated by the external application of energy such as light or ultrasonic energy, to facilitate targeted delivery to a region of the CNS. For example, in one embodiment, the LRP1 inhibitor is delivered in spherical lipid vesicles attached to gas-containing ultrasound-sensitive microbubbles, activated by focused ultrasound waves applied to the target region

In one embodiment, the LRP1 inhibitor is coated onto an implant or drug eluting device, such as a hydrogel or stent.

### EXAMPLES

### Example 1: LRP1 is a Master Regulator of Tau Uptake and Spread

Herein is shows that the low-density lipoprotein (LDL) receptor-related protein 1 (LRP1) controls tau endocytosis and subsequent spread. Knockdown of LRP1 significantly reduced tau uptake in H4 neuroglioma cells and iPS-derived neurons. The interaction between tau and LRP1 is mediated by lysine residues in the microtubule binding repeat region of tau. Furthermore, it was found that that downregulation of LRP1 in an in vivo mouse model of tau spread effectively reduced tau propagation between neurons. The results identify LRP1 as a key regulator of tau spread in the brain and, thus, as a novel target for diseases of tau spread and aggregation.

Results. Based on recent work highlighting the importance of heparan sulfate proteoglycans (HSPGs) in tau uptake, and the known role of LDL receptors to work in conjunction with HSPGs, it was to tested whether any of the LDL receptor family members could influence tau internalization. Using CRISPRi technology, gene expression of various LDLR family members was repressed (LRP1, LRP1B, LRP2, LRP5, LRP8, LDLR, VLDLR) in H4 neuroglioma cells. The ability of these cells to endocytose monomeric tau was assessed. Genetic silencing of LRP1 almost completely blocked the uptake of full-length soluble monomeric tau (2N4R isoform), whereas no other LDLR family member showed a significant effect. Different uptake mechanisms have been proposed for soluble and aggregated tau. LRP1 knockdown, surprisingly, was also sufficient to inhibit the uptake of tau oligomers, and reduced but did not completely inhibit the uptake of sonicated tau fibrils. Uptake of a disease-relevant mutant of tau and of phosphorylated tau were also affected by LRP1 knockdown. To show that LRP/knockdown was specific for tau endocytosis, transferrin uptake was analyzed in H4 cells and it was demonstrated that LRP/knockdown had no effect on the endocytosis of transferrin. Knockdown of LRP1 also prevented the uptake of the smaller isoforms of tau (0N3R, 0N4R, 1N3R, 1N4R, 2N3R) as well as fragments of tau that contain only the microtubule binding repeat region (K18; 4 repeats, K19; 3 repeats), highlighting the microtubule binding region as the potential interaction site.

To further support the results from the LRP1 knockdown cell lines, a well-known LRP1 binding protein, receptor-associated protein (RAP), was used as a competitor for tau uptake in wild-type (WT) H4 cells. RAP is a small 39 kDa chaperone for LRP1 that is known to bind tightly to LRP1 (K_{D}=9 nM). Increasing concentrations of RAP in the culture medium, concurrent with tau addition, were highly effective at inhibiting the uptake of both full-length and K18 tau (IC₅₀=4.9 nM and 9.6 nM, respectively), but did not influence the internalization of transferrin. Point mutations of key residues in RAP needed for its interaction with LRP1 (K256A, K270A—“mtRAP”) were sufficient to reduce this competitive effect.

In an attempt to understand how tau may interact with LRP1, the known crystal structures of the LDLR family bound to its ligands was examined. LRP1 (and other LDLR family members) contain cysteine-rich complement-type repeats (CRs) to bind and internalize their ligands. Each CR is composed of approximately 40 amino acids, six cysteines and an acidic residue cluster (normally aspartic acid) that coordinates Ca⁺² and interacts with lysine residues on ligands through salt bridges. Tau has a high lysine content, 44 lysines in 441 amino acids (10% content), and 20 of those are located within the microtubule binding region (K18: ˜ 15% content). Furthermore, cryo-EM structures of tau fibrillar aggregates from AD and CTE brains show 10 or 11 resolved lysines with all but one exposed to the exterior and thus available for interactions with other proteins. Therefore, to assess if lysine salt bridges with LRP1 were necessary for tau uptake all lysine residues on K18 were capped using a sulfo-NHS acetate and endocytosis was tested. Capping of lysine residues on K18 prevented uptake of tau in WT H4 cells, indicating that, similar to other LRP1 ligands, lysine residues are critical for this interaction.

LRP1 is a large (600 kDa) member of the LDLR family, and it contains 31 CR repeats divided into four different ligand binding domains. To determine which of these ligand binding domains influenced tau uptake, ectodomain constructs were designed, called mini-LRPs (mLRPs) to assess the ability of individual subdomains to rescue tau internalization in the CRISPRi LRP1 knockdown cells. By expressing these mLRPs in the absence of full length LRP1, it was found that full-length tau uptake was completely rescued with subdomain 4 (mLRP4) and partially rescued with mLRP2. Further, mLRP4 was able to co-immunoprecipitate tau confirming an interaction between the two. Only mLRP4 improved K18 uptake, indicating that two motifs in tau could possess the ability to bind LRP1, one within the microtubule binding region (amino acids 244-372) that interacts with mLRP4 and one in the N-terminal half (amino acids 1-243) or C-terminal end (amino acids 373-441) that interacts with mLRP2. To test this further, the uptake of the N-terminus of tau (amino acids 1-243) was examined and it as found that both mLRP4 and to a lesser extent, mLRP2 could rescue N-terminal uptake. This suggests a model where a primary interaction site on mLRP4 mediates tau uptake, but that the N-terminus of tau can also mediate interactions with a secondary site on mLRP2.

To determine if other known ligands of mLRP2 and mLRP4 are able to compete for uptake various ApoE isoforms were overexpressed in HEK293T cells and harvested the conditioned medium containing ApoE. Results showed that incubation of H4 cells in ApoE conditioned medium significantly reduced uptake of full-length tau, yet failed to reduce transferrin uptake.

Native LRP1 is highly expressed in neurons at the post-synaptic density, and previous work has highlighted that spread of tau in vivo is likely mediated trans-synaptically. Therefore, it was asked if uptake of tau in neurons was also regulated by LRP1. The expression of LRP1 in human iPS-derived neurons (iPSNs) was reduced using CRISPRi. Microscopy of adherent iPSNs showed that LRP1 knockdown efficiently reduced the amount of internalized tau. Quantification of tau endocytosis by flow cytometry confirmed the reduction of tau uptake upon LRP1 knockdown or upon addition of RAP, but not mtRAP into the culture medium. Similarly, to H4 cells, LRP1 knockdown or the addition of RAP had no effect on transferrin uptake in iPSNs.

Based on the foregoing in vitro results, it was sought to determine if LRP1 was also critical for tau spread in the brain. A recently developed model of tau spread has been described recently that utilizes an adeno-associated virus (AAV) and allows reliable discrimination of neurons that have been transduced to express human tau (hTau) versus neurons that receive hTau protein through spread, as described in Wegmann, S. et al. Experimental evidence for the age dependence of tau protein spread in the brain. Sci Adv 5, caaw6404, doi: 10.1126/sciadv.aaw6404 (2019). AAV encoding for one mRNA, GFP-P2A-hTau under control of a CMV promoter, but produces two proteins, GFP and hTau. The P2A peptide self-cleaves during translation and, thus, transduced neurons can be identified by the presence of GFP and hTau whereas cells that have taken up tau protein via spread mechanisms can be identified by the absence of GFP but the presence of hTau. To regulate LRP1 expression in parallel, an AAV (PHP.eB serotype) carrying an shRNA for LRP1 under the human synapsin (hSyn) promoter was used to knockdown LRP1 in neurons. AAVs coding either the LRP1 shRNA or a scramble control shRNA were retro-orbitally injected into six-week old wildtype mice, and two weeks later stereotactic injections of the AAV GFP-2A-hTau virus into the hippocampus were performed. After three weeks of incubation, the mice were sacrificed and spread was determined by immunofluorescence. Scramble and LRP1 shRNA AAVs contained the fluorescent protein reporter mRuby which allowed visualization of their expression throughout the brain. Animals expressing LRP1 shRNA showed reduced expression for LRP1 as determined by qPCR and IHC. To quantify tau spread, the number of hTau⁺/GFP⁻ cells were counted after immunostaining for human. A substantial amount of tau spread was observed in PBS and scramble injected animals (mean±s.d.=199±73 and 147±43 hTau⁺/GFP⁻ cells/mm², respectively), whereas spread was greatly diminished in LRP1 knockdown animals (19±19 hTau⁺/GFP⁻ cells/mm²). This was not due to differences in the number of transduced cells, as equivalent amount of GFP⁺ cells were observed across all animals. When analyzing tau spread by brain region, it was found that while LRP1 shRNA animals had similar numbers of spread cells in the ipsilateral hippocampus, spread was significantly diminished in the cortex. No effect of sex on the spread phenotype was observed. LRP1 is highly expressed in neurons, but it has also been shown that LRP1 is present in other cell types including astrocytes and microglia. Instances of hTau⁺ astrocytes were observed. Tau spread was also observed in the contralateral hippocampus in PBS and scramble injected mice, but was absent in the LRP1 shRNA animals.

Taken together, the study demonstrates that LRP1 is a master regulator of tau protein endocytosis in neurons, with an important role for tau spread in the brain. Targeting neuronal LRP1 led to a significant reduction of tau spread in vivo and provides a novel therapeutic approach for tau-related neurodegenerative diseases, targeting tau spread, and demonstrating LRP1 as a critical determinant for tau propagation.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

1.-17. (canceled)

18. A method of treating an individual to mitigate propagation of pathological tau between cells within the central nervous system, comprising:
administering to the individual a nucleic acid molecule that disrupts the expression of LRP1 within cells of the central nervous system by specifically targeting LRP1.

19. The method of claim 18, wherein the subject has, is suspected of having, or is at risk of a tauopathy selected from the group consisting of Alzheimer's disease, traumatic encephalopathy, corticobasal degeneration, Pick's disease, progressive supranuclear palsy, globular glial tauopathy, argyrophilic grain disease, and primary age-related tauopathy.

20. The method of claim 18, wherein the nucleic acid molecule is or encodes for expression of an antisense construct, a short interfering RNA, or a short hairpin RNA.

21. The method of claim 20, wherein the antisense molecule, the short interfering RNA, or the short hairpin RNA targets LRP1 RNA of the cells of the central nervous system.

22. The method of claim 20, the nucleic acid molecule is or encodes for expression of a short hairpin RNA.

23. The method of claim 22, wherein the nucleic acid molecule comprises a sequence such that has at least 90% identical to SEQ ID NO: 3.

24. The method of claim 23, wherein the nucleic acid molecule comprises a sequence such that has at least 95% identical to SEQ ID NO: 3.

25. The method of claim 24, wherein the nucleic acid molecule comprises a sequence such that has at least 99% identical to SEQ ID NO: 3.

26. The method of claim 18, wherein the nucleic acid molecule is CRISPR guide RNA.

27. The method of claim 26, wherein the CRISPR guide RNA targets LRP1 DNA of the cells of the central nervous system.

28. The method of claim 26, wherein the nucleic acid molecule comprises a sequence such that has at least 90% identical to SEQ ID NO: 5.

29. The method of claim 28, wherein the nucleic acid molecule comprises a sequence such that has at least 95% identical to SEQ ID NO: 5.

30. The method of claim 29, wherein the nucleic acid molecule comprises a sequence such that has at least 99% identical to SEQ ID NO: 5.

31. The method of claim 26, the CRISPR guide RNA is administered along with Cas9 or a nucleic acid construct for expressing Cas9.

32. The method of claim 18, wherein administering to the individual a nucleic acid molecule comprises delivery of the nucleic acid by a particle.

33. The method of claim 32, wherein the particle comprises one of: a liposome, amphiphilic block copolymer, a micelle, a PLGA nanoparticle, a poly(butylcyanoacrylate) nanoparticle, PEG-silica, bolaamphiphiles, chitosan, or PEG-polylactid.

34. The method of claim 18, wherein administering to the individual a nucleic acid molecule comprises delivery of the nucleic acid by a viral vector.

35. The method of claim 34, wherein the viral vector is adeno-associated virus.
